(19) United States
(12) Patent Application Publication
(10) Pub. No.: US 2025/0368947 A1
(43) Pub. Date: Dec. 4, 2025

(54) COMPOSITIONS COMPRISING SCLEROGLUCAN, ARTICLES CONTAINING THE SAME, METHODS OF MAKING, AND METHODS OF USING

(71) Applicant: NEOGEN FOOD SAFETY US HOLDCO CORPORATION, LANSING, MI (US)

(72) Inventors: Evan Douglas Brutinel, Lansing, MI (US); Alexi J. Young, Lansing, MI (US); Patrick A Mach, Lansing, MI (US); DUNCAN MINCKS, Lansing, MI (US); CARI K. LINGLE, Lansing, MI (US)

(21) Appl. No.: 18/878,054

(22) PCT Filed: Jul. 7, 2023
(86) PCT No.: PCT/US2023/069828

§ 371 Date: Dec. 22, 2024

(51) Int. Cl. C12N 1/20; C12M 1/00; C12M 1/12; C12N 1/22

(52) U.S. Cl. CPC C12N 1/20; C12M 23/38; C12M 25/04; C12M 37/00; C12M 37/04; C12N 1/22

(57) ABSTRACT

Compositions and articles comprising scleroglucan that can be used for culturing and enumerating microorganisms even after exposure to sterilizing doses of radiation. The compositions also include one or more water swellable polymers other than scleroglucan, such as guar gum, locust bean gum, or xantham gum. Also described are methods of making and using the same.

### CROSS-REFERENCE TO RELATED APPLICATIONS

This application claims priority to U.S. Application No. 63/367,956 filed on Jul. 8, 2022, the contents of which are hereby incorporated by reference in its entirety.

### FIELD OF INVENTION

The field of the invention relates generally to polymer films and the preparation and use thereof.

### BACKGROUND

Thin film culture devices have become quite popular over the last few decades. Thin film culture devices generally include a film positioned on a substrate. The film includes or acts as a culture media upon which microbial organisms can be grown. The films have a variety of uses, one of which is to assess the level of microbial contamination in a test sample.

U.S. Pat. No. 5,869,321 discloses thin film culture plates having medium particles comprising nutrients and a mixture of gelling agents. The gelling agents are materials such as carbohydrates, proteins and minerals, and specifically a mixture of xanthan gum, locust bean gum, and guar gum.

U.S. Pat. No. 9,988,600 discloses a dry powder cell culture medium with a polymer embedded component.

US20200109431 discloses a device for differentially enumerating colonies of coliform and Escherichia coli microorganisms. The device includes a first sheet with a first cold-water soluble gelling agent adhered to the first sheet as well as a second sheet with a second cold-water soluble gelling agent adhered to the second sheet. Guar gum, polyacrylamide, locust bean gum, and agar are mentioned as gelling agents, with guar gum and xanthan gum, alone or in combination, being preferred and guar gum being exemplified.

US20150225691 teaches a method of making a flowable, agglomerated nutrient medium using a fluidized bed agglomeration chamber. A gelling agent can be included. Binders are optionally included. Of the binders, PEG, polyvinyl pyrrolidone, polyvinyl alcohol, polysaccharide, dextran, dextrins, maltodextrins, microcrystalline cellulose, HPMC, methylcellulose, starch and sugars are mentioned.

U.S. Pat. No. 10,995,356 provides culture devices for enumerating colonies of microorganisms. Cold water-soluble gelling agent, dry buffer system, dry carbon dioxide generating system, and dry oxygen scavenging reagent are disposed in a growth compartment. Gelling agents mentioned include algin, carboxymethyl cellulose, tara gum, hydroxyethyl cellulose, guar gum, locust bean gum, xanthan gum, polyacrylamide, polyurethane, and polyethylene oxides. Guar gum, locust bean gum, and xanthan gum are preferred, either individually or in combination.

Feng et al. in Journal of Food Protection Vol. 80 (7) 2017 (1117-1122) disclose experiments “to determine potential enzymatic degradation of guar gum, the gelling agent used in Petrifilm™ plates.” The article concludes that liquefier organisms can hydrolyze the guar gum, which can have “two effects on the accuracy [of enumeration] (i) liquified areas may allow motile organisms to move and multiply . . . yielding more than one colony from one cell and as a result leading to overestimation of the microbial load and (ii) the blurred areas obscure other colonies, leading to potential underestimation.”

Scleroglucan is available from Cargill, Inc. (Minnetonka, MN USA) under the trade designation ACTIGUM and is said by its manufacturer to provide a stable viscosity over a pH range of 2.5-12 and a temperature between 10 and 120 degrees C. for use in construction, paint, home care, and detergents for drilling, oil recovery, and asphalt emulsion applications.

### SUMMARY

The present invention is directed towards compositions, articles, devices, and methods relating to thin films that include scleroglucan and one or more water swellable polymers.

In some embodiments, the present invention includes a composition comprises scleroglucan and one or more water swellable polymers other than scleroglucan.

In some embodiments, the present invention includes a culture device comprising a base member, a cover sheet connected to the base member, a growth compartment disposed between the cover sheet and the base member, and a scleroglucan layer that includes scleroglucan. In some embodiments, the scleroglucan layer is positioned within or adjacent to the growth compartment. In some other embodiments, the scleroglucan layer further includes one or more water swellable polymers other than scleroglucan.

In some embodiments, the present invention includes a method of culturing a microorganism. The methods of culturing can include contacting a microorganism with an inventive composition described herein to form an inoculated composition and allowing the microorganism to undergo at least one replication.

In some embodiments, the present invention includes a method of culturing a microorganism comprising contacting a microorganism with an inventive article described herein (e.g., a culture device described herein) to form an inoculated article and allowing the microorganism to undergo at least one replication.

In some embodiments, the present invention includes a method of sterilizing a culture device. The sterilization method can include providing an inventive article described herein (e.g., a culture device described herein), wherein the article includes one of the inventive compositions described herein. The sterilization method can further include the article to sufficient radiation such that the culture device is sterilized.

In some embodiments, the present invention includes a film comprising one of the inventive compositions described herein.

In some embodiments, the present invention includes an article comprising a substrate and an inventive film described herein.

### BRIEF DESCRIPTION OF THE DRAWINGS

FIG. 1 is a series of photographic images of Thin Film Culture Device CE-A (not exposed to radiation), Thin Film Culture Device CE-A (exposed to 30 kGy of radiation), and the Thin Film Culture Devices AA, BB, CC, and DD (exposed to 30 kGy of radiation) of Example 5 (Table 5) after inoculation with Escherichia coli (ATCC 25922) and incubation for 48 hours. For each type of thin film culture device, photographs of three separate devices are presented.

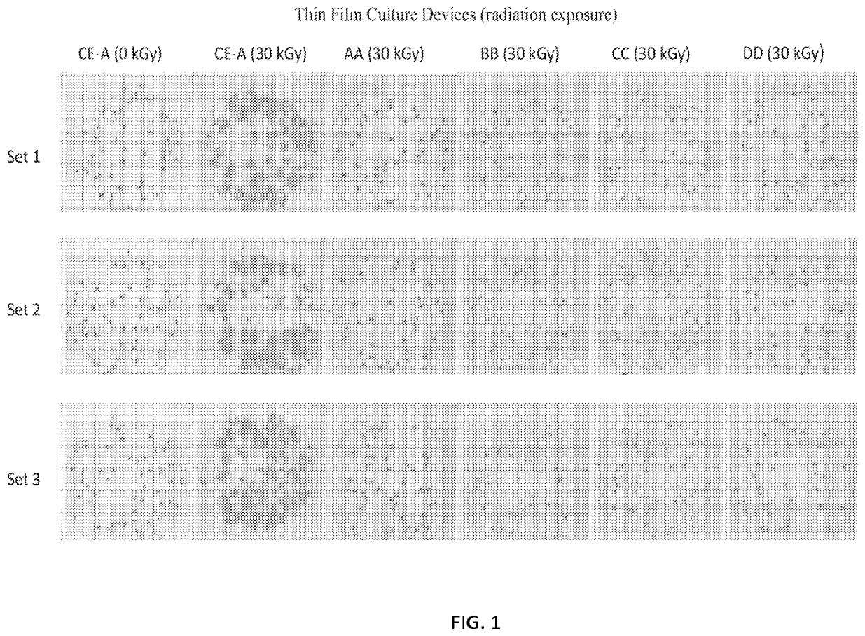

FIG. 2 is a series of photographic images of Thin Film Culture Device CE-A (not exposed to radiation), Thin Film Culture Device CE-A (exposed to 30 kGy of radiation), and the Thin Film Culture Devices AA, BB, CC, and DD (exposed to 30 kGy of radiation) of Example 5 (Table 5) after inoculation with Proteus mirabilis (ATCC 14153) and incubation for 48 hours. For each type of thin film culture device, photographs of three separate devices are presented.

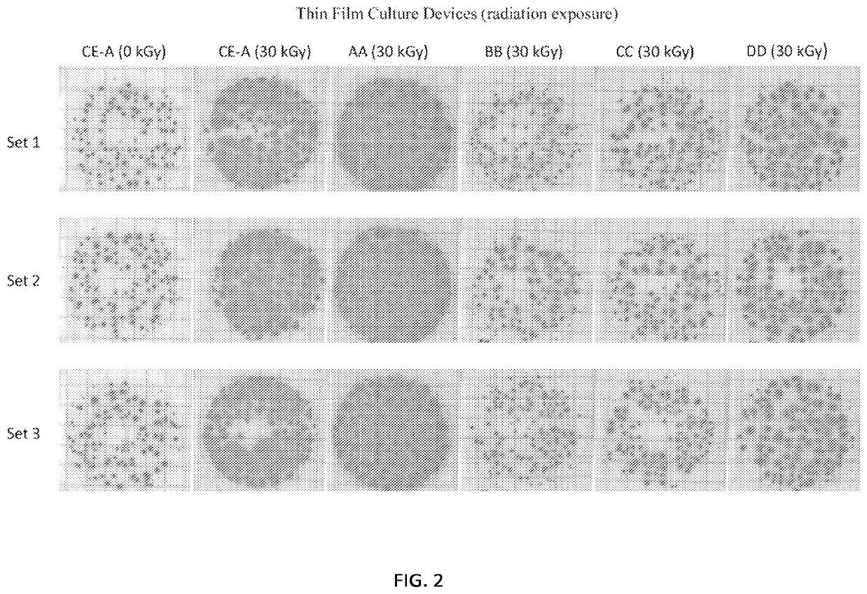

FIG. 3 is a series of photographic images of Thin Film Culture Device CE-A (not exposed to radiation), Thin Film Culture Device CE-A (exposed to 30 kGy of radiation), and the Thin Film Culture Devices AA, BB, CC, and DD (exposed to 30 kGy of radiation) of Example 5 (Table 5) after inoculation with Bacillus subtilis (ATCC 6633) and incubation for 48 hours. For each type of thin film culture device, photographs of three separate devices are presented.

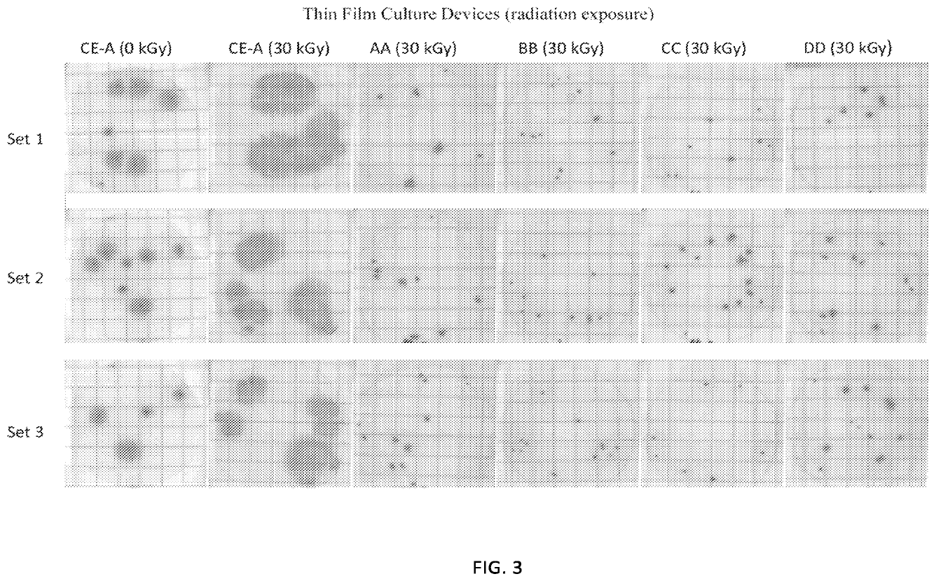

FIG. 4 is a series of photographic images of Thin Film Culture Device CE-A (not exposed to radiation), Thin Film Culture Device CE-A (exposed to 30 kGy of radiation), and the Thin Film Culture Devices EE, FF, GG, and HH (exposed to 30 kGy of radiation) of Example 5 (Table 6) after inoculation with Escherichia coli (ATCC 25922) and incubation for 48 hours. For each type of thin film culture device, photographs of three separate devices are presented.

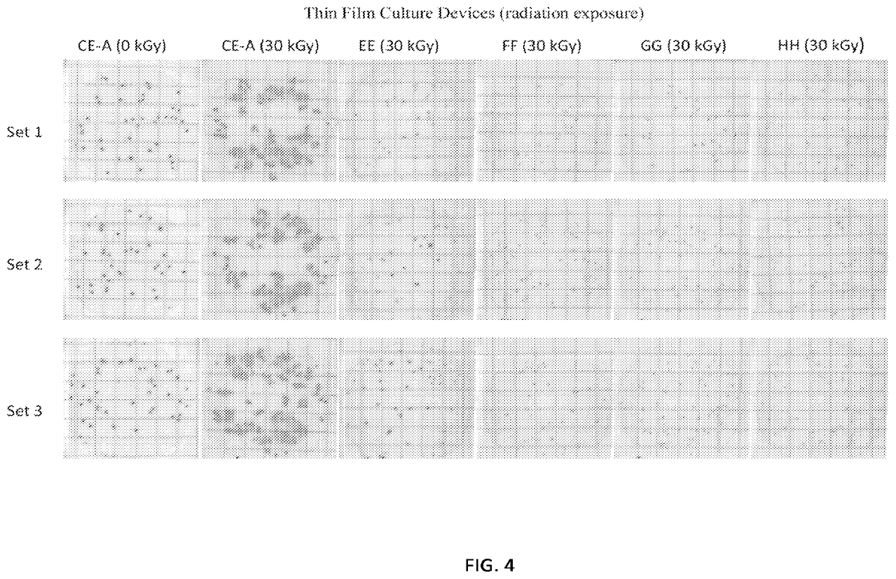

FIG. 5 is a series of photographic images of Thin Film Culture Device CE-A (not exposed to radiation), Thin Film Culture Device CE-A (exposed to 30 kGy of radiation), and the Thin Film Culture Devices EE, FF, GG, and HH (exposed to 30 kGy of radiation) of Example 5 (Table 6) after inoculation with Proteus mirabilis (ATCC 14153) and incubation for 48 hours. For each type of thin film culture device, photographs of three separate devices are presented.

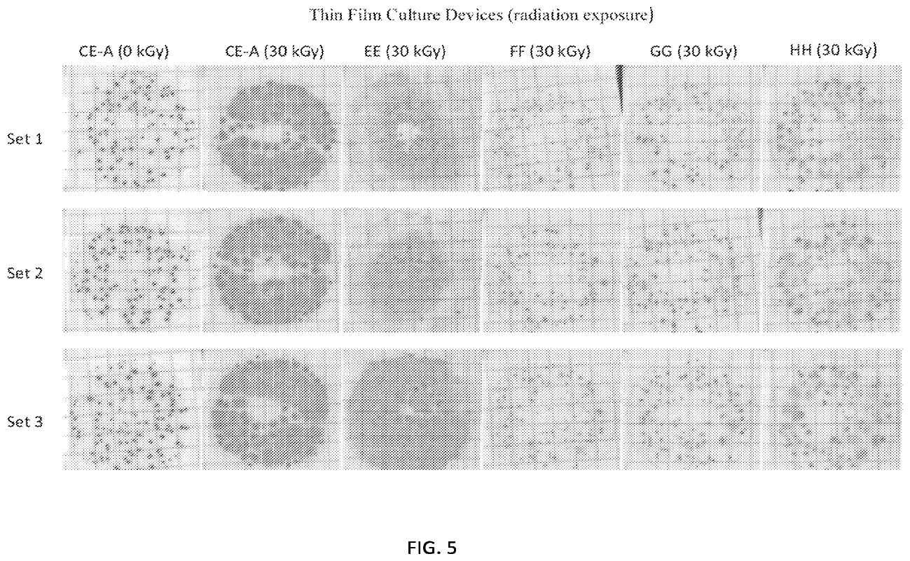

FIG. 6 is a series of photographic images of Thin Film Culture Device CE-A (not exposed to radiation), Thin Film Culture Device CE-A (exposed to 30 kGy of radiation), and the Thin Film Culture Devices EE, FF, GG, and HH (exposed to 30 kGy of radiation) of Example 5 (Table 6) after inoculation with Bacillus subtilis (ATCC 6633) and incubation for 48 hours. For each type of thin film culture device, photographs of three separate devices are presented.

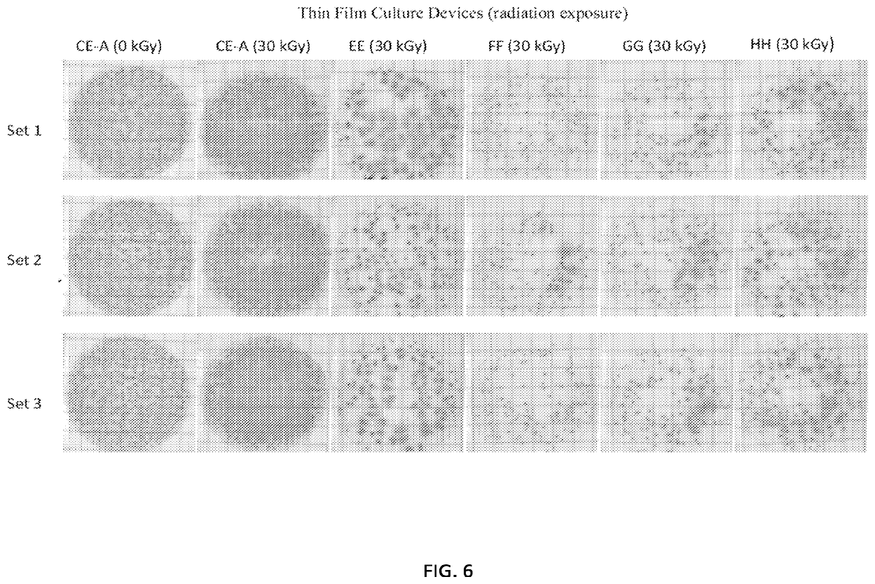

FIG. 7 illustrates one embodiment of an inventive article in the form of a culture device.

FIG. 8 illustrates a flow chart showing an embodiment of a method of the present invention used to manufacture or make a composition and article described herein.

FIG. 9 illustrates a flow chart showing an embodiment of a method of the present invention for using the compositions, layers, films, or articles described herein.

### DETAILED DESCRIPTION

In this application, terms such as “a,” “an,” and “the” are not intended to refer to only a singular entity but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

Terms such as “common,” “commonly,” “often,” “frequent,” and “frequently” are used to refer to features that are typically employed in the invention, but unless otherwise indicated are not meant to imply that the features so described were known or common before this disclosure.

The use of “or” means “and/or” unless stated otherwise.

The use of “comprise,” “comprises,” “comprising,” “include,” “includes” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Moreover, all ranges disclosed herein are to be understood to encompass any and all subranges (including all fractional and whole values) subsume therein.

The word “sterile” is used herein only as an adjective, and never as a verb. It may be used with or without reference to ISO 11137 or ISO11137-2:2013. Regardless of whether it is used with or without reference to ISO 11137 or ISO11137-2:2013, “sterile” means that the noun that it describes, such as a composition, film, article, or culture device, has a “negative test of sterility,” as that term is defined in ISO 11137-2:2013. The negative test of sterility may be established under either method 1 or method 2 of ISO 11137-2:2013; thus, an article that has a negative test of sterility when tested according to method 1 of ISO 11137-2:2013 and does not have a negative test of sterility when tested according to method 2 of ISO 11137-2:2013, or vice versa, or that has a negative test of sterility when tested according to both method 1 and method 2 of ISO 11137-2:2013, is considered “sterile” as that term is used herein.

The words “sterilize,” “sterilizes,” “sterilizing,” and “sterilized” are used herein only as verbs, and never as adjectives. Actions (i) to sterilize a thing (such as a composition, film, article, or culture device), (ii) that sterilizes a thing (such as a composition, film, article, or culture device), (iii) the sterilized thing (such as a composition, film, article, or culture device), or (iv) of sterilizing a thing (such as a composition, film, article, or culture device), all have the same meaning and differ only because of the grammatical requirements of the form of the verb “sterilize.” In each case, the verb (whether “sterilize,” “sterilizes,” “sterilizing,” or “sterilized”) means to make its object sterile (as the term “sterile” is defined above.) References to ISO 11137 or ISO 11137-2:2013 are to the English language version of ISO 111347-2:2013.

Thin film culture devices, such as those disclosed in, for example, U.S. Pat. Nos. 5,869,321, 9,988,600, and US202001943, include a substrate with a film that may incorporate a water swellable polymer, such as a gum (e.g., guar gum, xanthan gum, or locust bean gum). These water swellable polymers may provide a matrix in which a target microorganism may replicate and form colonies.

Exemplary thin film culture devices are available under the PETRIFILM™ brand name from Neogen Corporation, Lansing, MI USA. The instructions for use (“IFUs”) of PETRIFILM™ brand culture devices state that they are decontaminated though not sterilized.

This disclosure recognizes that prior art culture media and culture devices, such as those that contain one or more water swellable polymers such as one or more of guar gum, xanthan gum, and locust bean gum, and particularly thin film culture devices containing such polymers, could not be provided in a sterile state. The inventors attempted exposing such prior culture devices to doses of radiation that are sufficient to sterilize the devices, for e.g., at least 25 kGy or at least about 30 kGy, or about 25 to about 30 kGy; however, when using prior art culture devices that were exposed to sterilizing doses of radiation, the inventors found that the microorganisms being cultured could not be visualized as distinct colonies but rather formed patches. As a result, it was difficult or impossible to count the resulting colonies and accurately enumerate the number of colony-forming units (“cfus”) on the culture device.

While non-sterile culture devices that are decontaminated to reduce their bioburden, such as those sold under the PETRIFILM™ brand name, are acceptable for many applications, some applications such as medical diagnostics, clean room monitoring, and biopharmaceutical purification require culturing microorganisms under sterile conditions.

Thus, a problem can be stated as how to form a water swellable polymer composition that is suitable for use in as a culture media, for example as a component of a culture device, and that does not show unsuitably increased mobility of microorganisms after sterilization, particularly after exposure to at least about 25 kGy or at least about 30 kGy, or about 25 to about 30 kGy of radiation. Another problem can be stated as how to form a sterile composition of a water swellable polymer composition that is suitable for use in a culture device and that does not show unsuitably increased mobility of microorganisms.

Yet another problem can be stated as how to make a sterile film for use in microorganism culturing that includes a water swellable polymer composition and that does not show unsuitably increased mobility of microorganisms after sterilization, particularly after exposure to at least about 25 kGy or at least about 30 kGy, or about 25 to about 30 kGy of radiation.

Still another problem that can be stated is how to make a culture device, particularly a thin film culture device, that is sterile, for example that has been exposed to at least about 25 kGy or at least about 30 kGy of radiation, or about 25 to about 30 kGy, and that does not show unsuitably increased mobility of microorganisms.

In the context of these problems, when microbes form patches or splotches after incubation on a film or culture device, it can make it impossible to identify cfus or to count the number of colonies with reasonable accuracy.

Briefly, a solution to one or more of the aforementioned problems, and potentially to some other problems, involves the use of scleroglucan either alone or with other water swellable polymers, in the composition, film, or article for cell cultures.

Scleroglucan is a polysaccharide that is commercially available from Cargill, Inc. (Minnetonka, MN USA) under the trade designation ACTIGUM. Scleroglucan has a chemical structure that is similar to the chemical structure of polysaccharides, such as guar gum, xanthan gum, and locust bean gum. However, it has been surprisingly and unexpectedly discovered that scleroglucan can withstand irradiation without the problems mentioned above whereas polysaccharides generally cannot.

### Compositions

Compositions that can be used as culture media in culture devices, such as thin film culture devices, may have as their matrix scleroglucan alone or scleroglucan in combination with one or more additional water swellable polymers other than scleroglucan. The one or more additional water swellable polymers may include polymers that are at least partially soluble in water. For example, water soluble polymers that may be used in the compositions disclosed herein include those disclosed in U.S. Pat. Nos. 5,869,321, 9,988,600, or US20200109431 for use in water swellable or water gelling compositions. In some embodiments, when one or more additional water swellable polymers are used, they may comprise one or more of guar gum, xanthan gum, or locust bean gum. In further embodiments, a combination of locust bean gum and xanthan gum may be used.

When one or more additional water swellable polymers are used, the weight ratio of the scleroglucan to the one or more water additional swellable polymers may vary depending on the requirements of the final product. In some embodiments, the ratio of the total weight of the one or more water swellable polymers to the weight of the scleroglucan may be from about 1:99 to about 99:1, optionally from about 1:10 to about 10:1, and further optionally from about 3:1 to about 1:3. In further embodiments, the ratio of the total weight of the one or more water swellable polymers to the weight of the scleroglucan may be from about 10:90 to about 90:10, from about 20:80 to about 80:20, from about 25:75 to about 75:25, from about 30:70 to about 70:30, from about 40:60 to about 60:40, or from about 55:45 to about 45:55.

The amount of the one or more additional water swellable polymers can be characterized as a percentage of the sum of the total weight of the one or more additional water swellable polymers and the total weight of the scleroglucan. In some embodiments, the weight percent of the one or more additional water swellable polymers based on the sum of the total weight of the additional water swellable polymers and the total weight of scleroglucan may be about 1 or greater, optionally about 5 or greater, optionally about 10 or greater, optionally about 20 or greater, optionally about 25 or greater, optionally about 30 or greater, optionally about 33 or greater, optionally about 40 or greater, optionally about 50 or greater, optionally about 60 or greater, optionally about 67 or greater, optionally about 70 or greater, optionally about 75 or greater, optionally about 80 or greater, optionally about 90 or greater, or optionally about 95 or greater, or about 1 to about 95 wt %, or about 5 to about 75 wt %, or about 10 to about 50 wt % or about 5 to about 30 wt %.

The amount of the scleroglucan can also be characterized as a percentage of the sum of the total weight of the one or more additional water swellable polymers and the total weight of the scleroglucan. In some embodiments, the weight percent of the scleroglucan based on the sum of the total weight of the one or more additional water swellable polymers and the total weight of scleroglucan may be about 1 or greater, optionally about 5 or greater, optionally about 10 or greater, optionally about 20 or greater, optionally about 25 or greater, optionally about 30 or greater, optionally about 33 or greater, optionally about 40 or greater, optionally about 50 or greater, optionally about 60 or greater, optionally about 67 or greater, optionally about 70 or greater, optionally about 75 or greater, optionally about 80 or greater, optionally about 90 or greater, or optionally about 95 or greater, 100 wt % or about 1 to 100 wt % or about 25 to about 100 wt % or about 10 to about 70 wt %.

Additional components may be used in the compositions disclosed herein. Examples of such additional components include one or more nutrients that facilitate the growth of one or more microorganisms. In some embodiments, nutrients include one or more sugars, such as one or more of glucose, lactose, fructose, maltose, and dextrose, soluble starch, tryptone, proteose peptone, soytone, yeast extract, casamino acids, casein, pancreatic digest of casein, casein acid hydroxylase, papaic digest of soybean, sodium pyruvate, sodium chloride, magnesium chloride, magnesium sulfate, or dipotassium phosphate. In further embodiments, glucose may be used as an additional component.

Other additional components that may be used include selective agents (e.g., antibiotics), detection agents (for example dyes, such as redox dyes), carbon dioxide generating agents, and oxygen reducing agents.

In some embodiments, the composition may be a sterile composition, such as an irradiated composition, for example a composition that has been exposed to about 25 kGy or more of radiation or even about 30 kGy or more of radiation.

The composition may preferably meet the ISO 11737-1:2013 standard. In some embodiments, the composition may be disposed within a packaging, such as a sterile packaging, it may meet this standard while it may be in the packaging but it may not meet the standard once removed from its packaging because, for example, it may be contaminated soon after removal from its packaging.

### Articles

Another aspect of the invention pertains to articles comprising any of the compositions discussed herein. Coated and/or dried layers, such as films, that comprise any of the compositions discussed herein are also contemplated. An article may comprise a layer that is coated or dried, such as a film, which layer or film that in turn may comprise any of the compositions discussed herein. In some embodiments, the article may also comprise a substrate that may contact the layer or film. In further embodiments, the article may be sterile. The sterile article may be an irradiated article that has been exposed to 25 kGy or more of radiation, optionally 30 kGy or more of radiation.

The article may be in the form of a culture device, such as a culture plate. However, other forms may be possible; one example of another form may be a bag with the composition inside the bag.

In some embodiments, when the article may be a culture device, particularly a culture plate, it may have a base member, a cover sheet (sometimes referred to as a “cover slip”) over the base member, and a growth compartment between the cover sheet and the base member. The growth compartment may include any of the compositions described herein, for example scleroglucan without another water swellable polymer, or scleroglucan in combination with another one or more water swellable polymers. Additional components, such as those discussed above with reference to compositions, may also be included. In some embodiments, the cover sheet is movable, and optionally removable, to expose the growth compartment and to allow the growth compartment to be inoculated.

The composition including scleroglucan may be disposed on the base member, in which case the cover sheet may be disposed to cover the composition. In an alternative configuration, the composition or film may be disposed on the cover sheet. In another alternative configuration, the composition or film may be disposed on both the substrate and the cover sheet.

In some embodiments, a spacing member may be disposed between the base member and the cover sheet. When present, the spacing member may provide space for the growth compartment, such as, for example, the scleroglucan containing composition, which may also contain one or more additional components as discussed herein.

In some embodiments, an adhesive may be used. The adhesive may be disposed on the base layer, the cover sheet, or both, depending on the desired configuration of the device. Suitable adhesives include those known for use in thin film culture devices, such as acrylates (e.g., isooctyl acrylate based adhesives) or a mixture of one or more acrylates with an acrylamide.

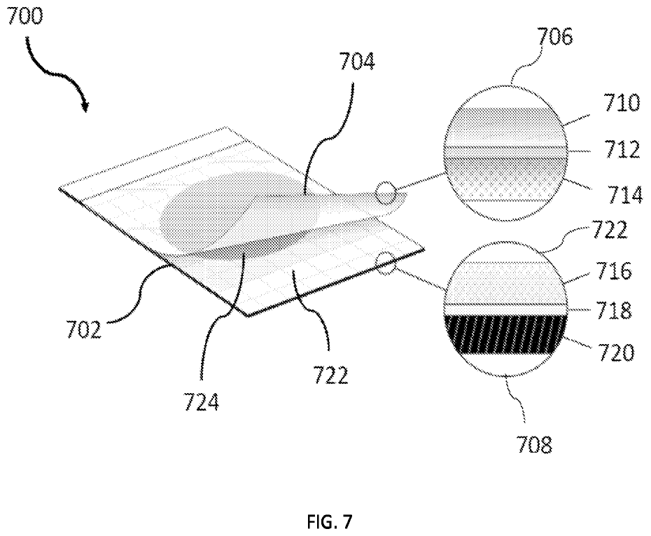

FIG. 7 illustrates one embodiment of an inventive article in the form of culture device 700. Culture device 700 includes base member 702 and cover sheet 704. Cover sheet 704 overlays one side of base member 702. FIG. 7 illustrates two magnified views of different portions of device 700. Magnified view 706 illustrates a cross-section view of cover sheet 704, while magnified view 708 illustrates a cross-section view of base member 702.

As shown in magnified view 706, cover sheet 704 includes at least three distinct layers: plastic film layer 710, adhesive layer 712, and inventive composition layer 714. Plastic film layer 710 is superior to both adhesive layer 712 and inventive composition layer 714. Plastic film layer 710 is impervious to water and is supple, allowing cover sheet 704 to be peeled away from base member 702. Adhesive layer 712 is disposed inferior to plastic film layer 710 and superior to inventive composition layer 714 and provides for improved adhesion between layers 710 and 714. Inventive composition layer 714 is inferior to both layers 710 and 712 and comprises one of the inventive scleroglucan-containing compositions described herein.

As shown in magnified view 708, base member 702 includes at least three distinct layers: plastic-coated paper layer 720, adhesive layer 718, and inventive composition layer 716. Plastic-coated paper layer 720 is inferior to both adhesive layer 718 and inventive composition layer 716. Plastic-coated paper layer 720 is impervious to water and provides base member 702 with a relatively rigid substrate from which cover sheet 704 can be to be peeled away or pressed against. Adhesive layer 718 is disposed superior to plastic-coated paper layer 720 and inferior to inventive composition layer 716 and provides for improved adhesion between layers 720 and 716. Inventive composition layer 716 is superior to both layers 718 and 720 and comprises one of the inventive scleroglucan-containing compositions described herein. Plastic-coated paper layer 720 is printed with a grid that allows a user to more easily count cfus during use of device 700.

Device 700 includes spacing member 722 which is a layer of plastic or plastic-coated paper disposed between the inferior surface of cover sheet 704 and the superior surface of base member 702. Cover sheet 704 acts as a spacer between cover sheet 704 and base member 702 and defines a circular or ovular space therebetween in the form of growth compartment 724. Growth compartment 724 is essentially a void defined superiorly by the inferior surface of inventive composition layer 714 of cover sheet 704, defined inferiorly by the superior surface of inventive composition layer 716 of base member 702, and defined laterally by spacing member 722. In some embodiments, one of the inventive compositions described herein is disposed within a growth compartment of an inventive culture device.

### Method of Making

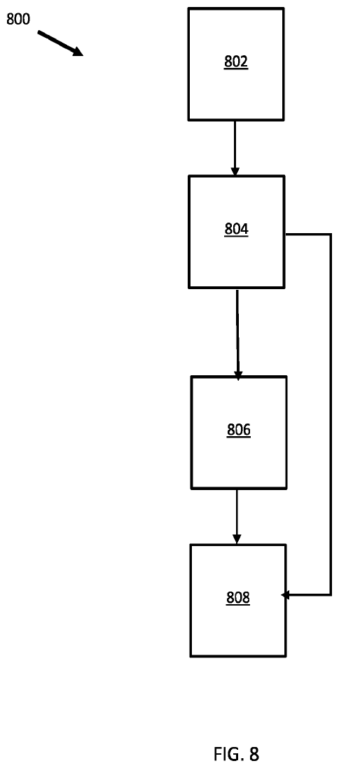

The compositions and the articles described herein may be made by any suitable method. FIG. 8 illustrates a flow chart showing an embodiment of a method of the present invention that can be used to manufacture or make a composition and article of the present invention in the form of method 800.

Step 802 of method 800 includes forming one of the inventive compositions described herein.

In some embodiments, the inventive compositions are made by admixing its components. For example, when the composition includes scleroglucan and one or more nutrients that facilitate the growth of one or more microorganisms, the components may be admixed either as powders or, in some embodiments, in the presence of a liquid to form a dispersion. Similarly, when the composition includes scleroglucan and one or more additional water swellable polymers, the components may be admixed either as powders or, in combination with a liquid to form a dispersion.

In some embodiments, when a liquid is used, it may be water, or it may be a combination of water and an organic solvent. When an organic solvent is used, it may be preferably selected so that the mixture of water and organic solvent may be a single phase. This may be accomplished, for example, by selecting a solvent that may be miscible with water such as a short-chained alcohol, like methanol, ethanol, or propanol, or ether like ethyl acetate, or some esters such as tetrahydrofuran, or by using a sufficiently low concentration of organic solvent such that the mixture of water and organic solvent may form only a single phase.

When the components are admixed as a liquid dispersion, the concentration of the components in the liquid will depend on the precise nature of the components that are used and the intended type of article. When a film is to be formed from the liquid, then the concentration of the scleroglucan and, if used, the one or more additional water swellable polymers, must be such that the viscosity of the dispersion is low enough that it can be manipulated and coated onto a substrate. If the concentrations are too high a gel will form, making it impossible to coat the solid components of the dispersion. If, however, a film is not to be formed, but instead the solid components and the liquid are to be used as a gel in an article, for example, as a gel in a culture bag, then it may be acceptable to use a concentration that forms a gel.

Step 804 of method 800 includes forming a film or layer from the inventive composition formed in Step 802. When a film or layer may be formed, the liquid may be coated on the desired substrate in any suitable manner. Examples include knife coating, die coating, spray coating, spin coating, and the like. After coating, the film may be partially or completely dried to remove all or part of the liquid. This may be accomplished by any known method, heating, exposure to reduced pressure, or by allowing the film or article to air-dry under ambient conditions.

In some embodiments, when a spray may be formed, such as when the components are mixed as powders and the powders sprayed onto the base member, cover sheet, or both, any known powder spraying or powder coating device may be used. In further embodiments, an adhesive may be applied to the cover sheet, the base layer, or both, in order to adhere the powder composition to the appropriate component or components of the device.

Step 806 of method 800 is optional and includes packaging a composition, film, or article of the present invention. In some embodiments, the package includes a barrier layer that seals the contents, including the film, article, or composition, from the exterior environment. Any packaging known for this purpose, for example packaging known for use with medical devices or PETRIFILM™ brand thin film culture plates, may be employed.

Step 808 of method 800 includes sterilizing the inventive composition, film or article. When the optional packaging step is carried out, the sterilization step may take place either before, concurrently with, or after the optional packaging step. In further embodiments, the sterilization step may occur concurrently with or after the optional packaging step so that the composition, film, or article can be protected by the packaging and remain sterile for a period of time, for example, until the packaging is opened.

A sterilization step may be used to make the composition, film, or article sterile. This may be accomplished by exposing the compositions, films, or articles to ionizing radiation, such as actinic radiation. A variety of radiation types may be used for this purpose such as gamma, electron beam (eBeam), and x-ray. In some embodiments, the compositions, films, or articles may be irradiated with actinic radiation, in order to make them sterile. Typically, at least 25 kGy of radiation is used. In some cases, at least 30 kGy of radiation is used.

Gamma irradiation may be generated using commercially available sources, such as those available from Atomic Energy of Canada, Inc., for example. Some such sources may use a cobolt-60 high-energy source. E-beam irradiation sources are also commercially available, such as the e-beam system available from ESI CB-300. X-ray machines are also well known and available to the artisan.

### Methods of Using

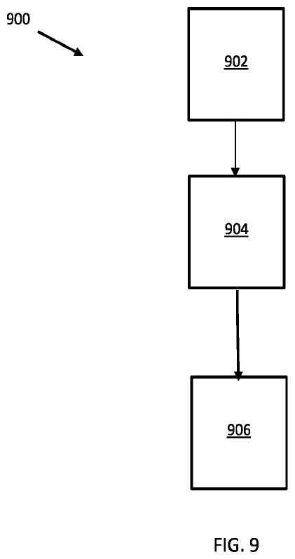

This invention further includes methods of using the compositions, layers, films, or articles disclosed herein to culture one or more microorganisms. FIG. 9 illustrates a flow chart showing an embodiment of a method of using the compositions, layers, films, or articles disclosed herein in the form of method 900.

At step 902 of method 900, a sample (e.g., an aqueous sample) containing one or more microorganisms may be contacted with the composition, layer, film, or article to form an inoculate composition, layer, film, or article.

At step 904 of method 900, the inoculated composition, layer, film, or article may then be allowed to incubate, at an elevated temperature for sufficient time to allow at least one replication of the one or more microorganisms. Exemplary elevated temperatures include from about 30° C. to about 80° C., such as about 30° C., about 32° C., about 37° C., about 40° C., about 42° C., about 45° C., about 50° C., about 60° C., or about 70° C., etc. Other temperatures might be used depending on the precise content of the composition, film, or article, the nature of the microorganism being cultured, and other factors known to artisans skilled in the relevant art. The required time may be about 30 minutes to about 72 hours, from about 1 hour to about 60 hours, from about 2 hours to about 50 hours, from about 5 hours to about 40 hours, from about 6 hours to about 30 hours, from about 7 hours to about 25 hours, or from about 8 to 24 hours. The required time may be from about 8 hours to about 10 hours, from about 10 hours to 12 hours, from about 12 hours to 14 hours, from about 14 hours to 16 hours, from about 16 hours to about 18 hours, from about 18 hours to about 20 hours, from about 20 hours to about 22 hours, or from about 22 hours to about 24 hours.

At optional step 906 of method 900, the composition, film, or article may optionally be enumerated to determine the number of cfus of the one or more microorganisms that may have formed. This may be done manually by human observation or by using a commercially available instrument, such as the PETRIFILM™ Plate Reader Advanced (3M Company, St Paul, MN, USA) that is designed for this purpose.

### EXAMPLES

### Materials

BACTO Tryptic Soy Broth (TSB) powder was obtained from Becton, Dickinson and Company, Franklin Lakes, NJ. The manufacturer reported the composition of the TSB powder to be: tryptone (57 weight %), soytone (10 weight %), glucose (8 weight %), sodium chloride (17 weight %), dipotassium phosphate (8 weight %).

R2A broth powder was obtained from HIMEDIA Laboratories (Mumbai, India). The manufacturer reported the composition of the R2A broth powder to be: casein acid hydroxylase (16 weight %); yeast extract (16 weight %); proteose peptone (16 weight %); dextrose (16 weight %); starch, soluble (16 weight %); dipotassium phosphate (9.6 weight %); magnesium sulphate (0.8 weight %); sodium pyruvate (9.6 weight %).

Scleroglucan was obtained from Cargill, Incorporated under the trade designation ACTIGUM CS-6 scleroglucan.

2,3,5-Triphenyl Tetrazolium Chloride (TTC) and sodium pyruvate were obtained from the MilliporeSigma Company, St. Louis, MO.

3M PETRIFILM Aerobic Count Plates were obtained from the 3M Company, Maplewood, MN and designated as Comparative Example A (CE-A).

Butterfield's Buffer was obtained from the 3M Company.

Xanthan gum and locust bean gum were obtained from the CP Kelco Company, Atlanta, GA.

Guar Gum (Meyprogat 150) was obtained from Danisco, Copenhagen, Denmark.

The bacterial strains Escherichia coli (ATCC 25922), Bacillus subtilis (ATCC 6633), were obtained from Microbiologics, St. Cloud, MN.

The bacterial strain Proteus mirabilis (ATCC 14153) was obtained from ATCC (American Type Culture Collection), Manassas, Va.

Unless otherwise noted, water was obtained from a MILLI-Q water purification system (EMD Millipore, Billerica, MA).

### Preparatory Example 1. Inoculum Preparation

Sterile TSB liquid media was prepared per the manufacturer's instructions (30 g of TSB powder per liter of purified water, pH 7.3±0.2) and sterilized using an autoclave. Cultures of each bacterial strain were individually prepared in sterile test tubes containing 9 mL of sterile TSB and incubated overnight at 30° C. in an orbital shaking incubator at 225 rpm (revolutions per minute). Each inoculum was prepared by serially diluting a single culture sample with Butterfield's Buffer. The Escherichia coli (ATCC 25922) and Proteus mirabilis (ATCC 14153) inoculums were serially diluted (10-fold dilutions) to a final 10-8 dilution sample. The Bacillus subtilis (ATCC 6633) inoculum was serially diluted to a final 10-7 dilution sample. When used to inoculate Thin Film Culture Devices AA-DD, the Bacillus subtilis (ATCC 6633) inoculum sample was supplemented with sodium pyruvate (2 g/L).

### Example 1. Preparation of the Cover Sheets of Thin Film Devices

Powder Compositions A-D were prepared from scleroglucan powder, xanthan gum powder, and locust bean gum powder. Powder Composition A contained only scleroglucan powder. Powder Compositions B-D were powder mixtures that contained varying weight percent (wt. %) concentrations of scleroglucan powder, xanthan gum powder, and locust bean gum powder. For each powder mixture the components were combined and mixed by shaking in a plastic bag for 30 seconds to form a homogeneous mixture. The ratio of the powder components in the compositions is summarized in Table 1.

The cover sheet component of each thin film device was prepared starting with a biaxially-oriented polypropylene (BOPP) film (1.6 mil thick) having a pressure sensitive adhesive (96:4 weight ratio of isooctyl acrylate:acrylamide) at a coat weight of 1.3 mg/cm². The adhesive contained 0.1 weight percent of TTC based on the dry weight of adhesive. The adhesive coated surface of the film was powder coated with a single homogeneous powder mixture selected from Powder Compositions A-D. Each powder composition was evenly applied to the adhesive surface and excess powder was removed from the adhesive layer by hand shaking of the film. The resulting powder coated films were cut into 76 mm wide by 100 mm long sections to form thin film device cover sheets.

TABLE 1

| Powder | Components of the Composition (wt. %) | | |
|---|---|---|---|
| Composition | Scleroglucan | Xanthan Gum | Locust Bean Gum |
| A | 100 | 0 | 0 |
| B | 25 | 37.5 | 37.5 |
| C | 50 | 25 | 25 |
| D | 75 | 12.5 | 12.5 |

### Example 2. Preparation of the Base Member Components of Thin Film Culture Devices Containing TSB Powder as the Nutrient

The base member component of each thin film device was prepared starting with a clear, polyethylene coated paper sheet (1 cm×1 cm in size, 0.15 mm thick, coated on both sides). The paper sheet component was white with yellow grid lines (1 cm×1 cm). One side of the polyethylene coated paper sheet was coated with a pressure sensitive adhesive (98:2 weight ratio of isooctyl acrylate:acrylic acid) at a coat weight of 1.3 mg/cm². The adhesive coated surface was then powder coated with a single powder mixture selected from Powder Compositions E-H (Table 2). For each powder mixture the components were combined and mixed by shaking in a plastic bag to form a homogeneous mixture. Each powder composition was evenly applied to the adhesive surface and excess powder was removed from the adhesive layer by hand shaking of the film. The resulting powder coated films were cut into 76 mm wide by 100 mm long sections to form thin film device base member components.

TABLE 2

| | Components of the Composition (wt. %) | | | |
|---|---|---|---|---|
| Powder | | Xanthan | Locust | |
| Composition | Scleroglucan | Gum | Bean Gum | TSB Powder |
| E | 67 | 0 | 0 | 33 |
| F | 16.5 | 24.75 | 24.75 | 34 |
| G | 33 | 16.5 | 16.5 | 34 |
| H | 49.5 | 8.25 | 8.25 | 34 |

### Example 3. Preparation of the Base Member Components of Thin Film Culture Devices Containing R2A Broth Powder as the Nutrient

Base member components were individually prepared from Powder Compositions I-L (Table 3) using the same procedure as described in Example 2 with the exception that TSB powder was replaced with R2A broth powder in the mixtures used.

TABLE 3

| | Components of the Composition (wt. %) | | | |
|---|---|---|---|---|
| Powder | | Xanthan | Locust | |
| Composition | Scleroglucan | Gum | Bean Gum | R2A Powder |
| I | 67 | 0 | 0 | 33 |
| J | 16.5 | 24.75 | 24.75 | 34 |
| K | 33 | 16.5 | 16.5 | 34 |
| L | 49.5 | 8.25 | 8.25 | 34 |

### Example 4. Preparation of Thin Film Devices

Thin film culture devices were assembled by attaching a cover sheet selected from Example 1 to a base member component selected from Examples 2 and 3. Each cover sheet was attached to a base member (in a hinge-like manner) along one edge (the 76 mm edge) using a strip of double sided adhesive tape (about 9.5 mm wide). For each device, the cover sheet and the base member were oriented so that the edges were aligned and the powder coated surface of the cover sheet faced the powder coated surface of the base member. The construction of the Thin Film Culture Devices AA-HH is summarized in Table 4.

TABLE 4

| | Powder | | Powder | | Nutrient |
|---|---|---|---|---|---|
| Thin Film | Composition | | Composition | | in Powder |
| Culture | of Cover Sheet | | of Base Member | | Composition of |
| Device | (% Scleroglucan) | | (% Scleroglucan) | | Base Member |
| AA | A | (100%) | E | (67%) | TSB |
| BB | B | (25%) | F | (16.5%) | TSB |
| CC | C | (50%) | G | (33%) | TSB |
| DD | D | (75%) | H | (49.5%) | TSB |
| EE | A | (100%) | I | (67%) | R2A |
| FF | B | (25%) | J | (16.5%) | R2A |
| GG | C | (50%) | K | (33%) | R2A |
| HH | D | (75%) | L | (49.5%) | R2A |

### Example 5. Inoculation, Incubation, and Colony Observation of Thin Film Devices

Thin Film Culture Devices AA-HH were exposed to 30 kGy of gamma radiation. The irradiated Thin Film Culture Devices AA-HH of Example 4 were inoculated with a single inoculum of either Escherichia coli (ATCC 25922), Bacillus subtilis (ATCC 6633), or Proteus mirabilis (ATCC 14153). The devices were placed on a flat, horizontal surface. The cover sheet of each device was lifted and 1 mL of a single inoculum (i.e., final dilution described in Preparatory Example 1) was carefully added by pipette as a single application to a compact region in the center of the coated base member. The cover sheet was gently returned to its original position. A 3M PETRIFILM Yeast and Mold Spreader (obtained from the 3M Company) was applied to the external surface of the cover sheet with hand pressure to spread the inoculum so that it formed a circular region having a diameter of about 2⅜ inches (6.03 cm). The spreader was then removed and the plate was maintained undisturbed for 1-5 minutes to allow for gel formation. Inoculated devices were prepared in triplicate (n=3 for each of Devices AA-HH). Each device was then incubated at 32° C. for 48 hours. The same inoculation and incubation procedure was also conducted with 3M PETRIFILM Aerobic Count Plates (CE-A) that had been exposed to 30 kGy dose of gamma radiation and with 3M PETRIFILM Aerobic Count Plates (CE-A) that had not been irradiated.

Images of the devices were taken using a 3M PETRIFILM Advanced Plate Reader (obtained from the 3M Company and operated according to the manufacturer's instructions) and exported as JPEG image files. ImageJ software (National Institutes of Health, Bethesda, MD) was used to measure the areas of the red colored colonies present in the devices. Once an image file was opened, the scale of the image was set using an internal 10 mm standard with selection of the “Global” setting. For each image, the distance between the yellow grid lines in the base member was used to set the image scale. Next, the “Image-Adjust-Color Balance” setting was selected followed by selecting the setting “Yellow” from the drop down menu. The minimum and maximum slide bars were used to increase the contrast of the colony edges. The images were then converted to 8-bit and a threshold was set to select only the pixels representing colonies. In the “Analyze Particles” menu (within the software “Analyze” menu), small non-colony particles were eliminated from the analysis by visually identifying the smallest bacterial colony in the image and setting the area of that colony as the minimum size for colony identification by the software. The “OK” button was selected, and the areas (mm²) of the colonies were measured by the software program. The results are reported in Tables 5 and 6. For Escherichia coli (ATCC 25922) and Proteus mirabilis (ATCC 14153) samples, the average colony size was determined based on the analysis of 25-100 colony forming units (cfu) per device. For Bacillus subtilis (ATCC 6633) samples the average colony size was determined based on the analysis of 5-10 colony forming units (cfu) per device.

The designation “ND” in Tables 5 and 6 indicates that the borders of multiple colonies merged together resulting in large, diffuse, and/or irregularly shaped patches that could not be counted for the purpose of colony enumeration.

TABLE 5

| | | Average Colony Size Observed (mm²) | | |
|---|---|---|---|---|
| | Gamma | Escherichia | Proteus | Bacillus |
| Thin Film | Irradiation | coli | mirabilis | subtilis |
| Culture | Exposure | (ATCC | (ATCC | (ATCC |
| Device | (kGy) | 25922) | 14153) | 6633) |
| Comparative | 0 | 0.8 | 2.9 | 41.7 |
| Example A | | | | |
| (CE-A) | | | | |
| Comparative | 30 | ND | ND | 140.2 |
| Example A | | | | |
| (CE-A) | | | | |
| AA | 30 | 1.1 | ND | 4.1 |
| BB | 30 | 0.6 | 3.9 | 1.2 |
| CC | 30 | 0.7 | 5.7 | 2.8 |
| DD | 30 | 0.8 | 8.2 | 4.3 |

TABLE 6

| | | Average Colony Size Observed (mm²) | | |
|---|---|---|---|---|
| | Gamma | Escherichia | Proteus | Bacillus |
| Thin Film | Irradiation | coli | mirabilis | subtilis |
| Culture | Exposure | (ATCC | (ATCC | (ATCC |
| Device | (kGy) | 25922) | 14153) | 6633) |
| Comparative | 0 | 1.1 | 1.8 | 0.7 |
| Example A | | | | |
| (CE-A) | | | | |
| Comparative | 30 | 11.6 | ND | ND |
| Example A | | | | |
| (CE-A) | | | | |
| EE | 30 | 0.5 | ND | 2.6 |
| FF | 30 | 0.2 | 0.6 | 0.4 |
| GG | 30 | 0.3 | 1.0 | 1.0 |
| HH | 30 | 0.2 | 4.3 | 1.8 |

### LIST OF EMBODIMENTS

The following is a non-limiting list of embodiments:

1. A composition comprising:
a. one or more water swellable polymer other than scleroglucan, the one or more water swellable polymer optionally comprising one or more of guar gum, locust bean gum, or xanthan gum, and
b. scleroglucan (may be present in a weight percent based on the total weight of the one or more water swellable polymer other than scleroglucan and the total weight of scleroglucan, in the amount of about 1 or greater, optionally about 5 or greater, optionally about 10 or greater, optionally about 20 or greater, optionally about 25 or greater, optionally about 30 or greater, optionally about 33 or greater, optionally about 40 or greater, optionally about 50 or greater, optionally about 60 or greater, optionally about 67 or greater, optionally about 70 or greater, optionally about 75 or greater, optionally about 80 or greater, optionally about 90 or greater, or optionally about 95 or greater, 100 wt % or about 1 to 100 wt % or about 25 to about 100 wt % or about 10 to about 70 wt %).

2. A device comprising:
a. a base member;
b. a cover sheet connected to the base member; and
c. a growth compartment disposed between the cover sheet and the base member;
d. wherein the cover sheet can be moved to expose the growth compartment; and
e. further wherein the growth compartment comprising a composition of embodiment 1.

3. The composition or device of any of the preceding embodiments, further comprising one or more nutrients that facilitate the growth of one or more microorganisms.

4. The composition or device of any of the preceding embodiments, wherein the ratio of (i) the one or more water swellable polymer and (ii) the weight of the scleroglucan is 1:99 to 99:1, optionally 1:10 to 10:1, and further optionally 3:1 to 1:3.

5. The composition or device of any of the preceding embodiments, wherein the weight percent of the water swellable polymer based on the sum of the total weight of the water swellable polymer and the total weight of scleroglucan is 1 or greater, optionally 5 or greater, optionally 10 or greater, optionally 20 or greater, optionally 25 or greater, optionally 30 or greater, optionally 33 or greater, optionally 40 or greater, optionally 50 or greater, optionally 60 or greater, optionally 67 or greater, optionally 70 or greater, optionally 75 or greater, optionally 80 or greater, optionally 90 or greater, or optionally 95 or greater.

6. The composition or device of any of the preceding embodiments, wherein the weight percent of the scleroglucan based on the sum of the total weight of the water swellable polymer and the total weight of scleroglucan is scleroglucan is 95 or less, optionally 90 or less, optionally 80 or less, optionally 75 or less, optionally 70 or less, optionally 67 or less, optionally 60 or less, optionally 50 or less, optionally 40 or less, optionally 33 or less, optionally 30 or less, optionally 25 or less, optionally 20 or less, optionally 10 or less, optionally 5 or less, or optionally 1 or less.

7. The composition or device of any of the preceding embodiments comprising scleroglucan and one or more nutrients that facilitate the growth of one or more microorganisms.

8. The composition or device of any of the preceding embodiments, wherein the one or more nutrients include one or more of sugars, wherein the sugar optionally includes at least one of glucose, lactose, fructose, maltose, or sucrose, soluble starch, tryptone, soytone, yeast extract, casamino acids, sodium pyruvate, sodium chloride, magnesium chloride, magnesium sulfate, or dipotassium phosphate.

9. The composition or device of any of the preceding embodiments, wherein the one or more nutrients include glucose.

10. The composition or device of any of the preceding embodiments, wherein the composition or device is an irradiated composition or device that has been exposed to 25 kGy or more of radiation, optionally 30 kGy or more of radiation.

11. The composition or device of any of the preceding embodiments that is sterile.

12. The composition or device of any of the preceding embodiments, that is disposed within an interior a packaging, optionally wherein the packaging provides a sterile barrier between the interior of the packaging and an external environment.

13. An article comprising the composition or device of any of the preceding embodiments, optionally wherein the article is a culture device, optionally a thin film culture device.

14. The article of embodiment 13, wherein the article is an irradiated article that has been exposed to 25 kGy or more of radiation, optionally 30 kGy or more of radiation, or about 25 to about 35 kGy.

15. The article of any one of embodiments 13-14 that is sterile.

16. The article of any one of embodiments 13-15, that is disposed within an interior a packaging, optionally wherein the packaging provides a sterile barrier between the interior of the packaging and an external environment.

17. A film comprising the composition of any one of embodiments 1-12.

18. An article comprising a substrate and the film of embodiment 17.

19. A culture device comprising the film of any one of embodiments 17-18, wherein the culture device comprises a growth compartment defined by a backing and a cover member covering the growth compartment, and wherein the film is disposed on the backing, the cover member, or both.

20. A composition of any one of embodiments 1-13 for use in culturing one or more microorganisms.

21. A film of embodiment 17 for use in culturing one or more microorganisms.

22. An article of any one of embodiments 14-16 or 18-20 for use in culturing one or more microorganisms.

23. A method of culturing a microorganism comprising
a. contacting a microorganism with a composition of any one of embodiments 1-13 to form an inoculated composition, and
b. allowing the microorganism to undergo at least one replication.

24. A method of culturing a microorganism comprising
a. contacting a microorganism with a device of any one of embodiments 2-12 or an article of any one of embodiments 13-15 to form an inoculated article, and
b. allowing the microorganism to undergo at least one replication.

25. A method of culturing a microorganism comprising
a. contacting a microorganism with a film of embodiment 17 or an article of any one of embodiments 18-22 to form an inoculated article, and
b. allowing the microorganism to undergo at least one replication,
c. wherein the microorganism is optionally a microorganism of the genus Bacillus and further optionally Bacillus sp.

26. The method of any one of embodiments 24-25, further comprising incubating the inoculated composition or article, optionally at a temperature of 25 degrees C. to 80 degrees C. for 30 minutes to 72 hours.

27. The method of any one of embodiments 24-26, further comprising the steps of allowing the microorganism to form one or more microorganism colonies and counting the number of microorganism colonies.

28. A method of making a composition of embodiment 7, comprising admixing scleroglucan with one or more one or more nutrients that facilitate the growth of one or more microorganisms and optionally a water swellable polymer other than scleroglucan.

29. A method of sterilizing a composition or device of any one of embodiments 1-14, comprising exposing the composition or device to sufficient radiation such that the composition is sterile.

30. The method of embodiment 28, further comprising exposing the composition to sufficient radiation such that the composition is sterile.

31. The method of any one of embodiments 28-30, further comprising exposing the composition to at least 25 kGy of irradiation, optionally at least 30 kGy of irradiation.

32. The method of any one of embodiments 28-31, wherein the water swellable polymer other than scleroglucan comprises one or more of guar gum, locust bean gum, and xanthan gum, and optionally locust bean gum, xanthan gum, or a mixture of locust bean and xanthan gums.

33. The method of any one of embodiments 28-31, wherein the ratio of (i) the one or more water swellable polymer and (ii) the weight of the scleroglucan is 1:99 to 99:1, optionally 1:10 to 10:1, and further optionally 3:1 to 1:3.

34. The method of any one of embodiments 28-32, wherein the weight percent of the water swellable polymer based on the total weight of the water swellable polymer and the total weight of scleroglucan is 1 or greater, optionally 5 or greater, optionally 10 or greater, optionally 20 or greater, optionally 25 or greater, optionally 30 or greater, optionally 33 or greater, optionally 40 or greater, optionally 50 or greater, optionally 60 or greater, optionally 67 or greater, optionally 70 or greater, optionally 75 or greater, optionally 80 or greater, optionally 90 or greater, or optionally 95 or greater.

35. The method of any one of embodiments 28-31, wherein the weight percent of the water swellable polymer based on the total weight of the water swellable polymer the total weight of scleroglucan is scleroglucan is 95 or less, optionally 90 or less, optionally 80 or less, optionally 75 or less, optionally 70 or less, optionally 67 or less, optionally 60 or less, optionally 50 or less, optionally 40 or less, optionally 33 or less, optionally 30 or less, optionally 25 or less, optionally 20 or less, optionally 10 or less, optionally 5 or less, or optionally 1 or less.

36. A method of making a sterile culture device, comprising
a. fixing a composition of any one of embodiments 1-13 on a substrate to form a culture device; and
b. exposing the culture device to sufficient radiation to make the composition sterile.

37. The method of embodiment 36, comprising exposing the composition to at least 25 kGy of irradiation, optionally at least 30 kGy of irradiation, or about 25 to about 35 kGy.

38. The method of any one of embodiments 36-37, further comprising placing the culture device in a packaging.

39. The method of embodiment 38, wherein the step of exposing the culture device to radiation occurs while the culture device is disposed in the packaging.

40. The method of any one of embodiments 38-39, wherein the step of exposing the culture device to radiation occurs while the culture device is sealed in the packaging.

41. The composition or device of any of the preceding embodiments, wherein scleroglucan is present in a weight percent of the scleroglucan based on the total weight of the one or more additional water swellable polymers and the total weight of scleroglucan may be about 1 or greater, optionally about 5 or greater, optionally about 10 or greater, optionally about 20 or greater, optionally about 25 or greater, optionally about 30 or greater, optionally about 33 or greater, optionally about 40 or greater, optionally about 50 or greater, optionally about 60 or greater, optionally about 67 or greater, optionally about 70 or greater, optionally about 75 or greater, optionally about 80 or greater, optionally about 90 or greater, or optionally about 95 or greater, 100 wt % or about 1 to 100 wt % or about 25 to about 100 wt % or about 10 to about 70 wt %.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

All publications mentioned herein are incorporated by reference to the extent they support the present invention.

1-27. (canceled)

28. A composition comprising:
(i) one or more water swellable polymers other than scleroglucan, and
(ii) scleroglucan.

29. The composition of claim 28, wherein the one or more water swellable polymers include one or more of guar gum, locust bean gum, or xanthan gum.

30. The composition of claim 28, wherein the weight ratio of the one or more water swellable polymers to the scleroglucan is 1:99 to 99:1.

31. The composition of claim 28, wherein the weight percent of the one or more water swellable polymers based on the sum of the total weight of the one or more water swellable polymers and the total weight of the scleroglucan is 1 or greater.

32. The composition of claim 28, further including one or more nutrients that facilitate the growth of one or more microorganisms.

33. The composition of claim 32, wherein the one or more nutrients include a sugar, a soluble starch, tryptone, soytone, yeast extract, casamino acids, sodium pyruvate, sodium chloride, magnesium chloride, magnesium sulfate, or dipotassium phosphate.

34. The composition of claim 28, wherein the composition is an irradiated composition that has been exposed to 25 kGy or more of radiation.

35. The composition of claim 28, wherein the composition is sterile.

36. A culture device comprising:
a base member;
a cover sheet connected to the base member;
a growth compartment disposed between the cover sheet and the base member; and
a scleroglucan layer that includes scleroglucan, the scleroglucan layer positioned within or adjacent the growth compartment.

37. The culture device of claim 36, wherein the scleroglucan layer further includes one or more water swellable polymers other than scleroglucan.

38. The culture device of claim 36, wherein the one or more water swellable polymers include one or more of guar gum, locust bean gum, or xantham gum.

39. The culture device of claim 36, wherein the weight ratio of the one or more water swellable polymers to the scleroglucan in the scleroglucan layer is 1:99 to 99:1.

40. The culture device of claim 36, wherein the weight percent of the one or more water swellable polymers based on the sum of the total weight of the one or more water swellable polymers and the total weight of the scleroglucan is 1 or greater.

41. The culture device of claim 36, wherein the scleroglucan layer further includes one or more nutrients that facilitate the growth of one or more microorganisms.

42. The culture device of claim 41, wherein the one or more nutrients include a sugar, a soluble starch, tryptone, soytone, yeast extract, casamino acids, sodium pyruvate, sodium chloride, magnesium chloride, magnesium sulfate, or dipotassium phosphate.

43. The culture device of claim 36, wherein the culture device has been sterilized by exposure to 25 kGy or more of radiation.

44. The culture device of claim 36, further including a packaging, wherein the culture device is disposed within an interior of the packaging and wherein the packaging provides a sterile barrier between the culture device and an external environment.

45. A method of culturing a microorganism comprising:
contacting a microorganism with a composition of any one of claims 1-10 to form an inoculated composition; and
allowing the microorganism to undergo at least one replication.
